(19) United States
(12) Patent Application Publication
(10) Pub. No.: US 2004/0131673 A1
(43) Pub. Date: Jul. 8, 2004

(54) Manufacturing dissolvable dosage forms

(76) Inventors: Ronald Alan Coffee, Surrey (GB); David Neville Davies, Oxford (GB); Johnathan Essex-Lopresti, Berkshire (GB); Margaret Sin Ka Wan, Oxfordshire (GB); Anna Busby, Oxfordshire (GB)

Correspondence Address: Frost Brown Todd, 2200 PNC Center, 201 East Fifth Street, Cincinnati, OH, 45202, US

(21) Appl. No.: 10/472,588

(22) PCT Filed: Mar. 22, 2002
(86) PCT No.: PCT/GB02/01404

(30) Foreign Application Priority Data
Mar. 22, 2001 (GB) 0107225.5
Apr. 3, 2001 (GB) 0108340.1
Jun. 15, 2001 (GB) 0114674.5
Jul. 4, 2001 (GB) 0116363.3
Sep. 7, 2001 (GB) 0121677.9
Sep. 17, 2001 (GB) 01222399.9

(51) Int. Cl. A61K 9/20

(52) U.S. Cl. 424/465

(57) ABSTRACT

A method of manufacturing a dosage form is described wherein a liquid solution of a biologically compatible polymer containing a suspension of particulate material that is insoluble in the polymer is supplied to a liquid supply tube (2) having an outlet (2a) and an electrical field is established between the outlet (2a) and a surface (19) spaced from th outlet (2a) to cause liquid issuing from the outlet to form a jet which dries to form a polymer fibre containing particles of the particulate material and which deposits onto the surface (19) to form a dosage which consists of said particulate containing fibre which fibre dissolves or disintegrates in a wet environment such as the mouth.

This invention relates to the manufacturing of dissolvable dosage forms especially, but not exclusively, dissolvable dosage forms carrying at least one pharmacologically or biologically active ingredient for therapeutic or prophylactic treatment of an animal such as a human being.

Conventional medicines to be ingested in a solid form are manufactured as either a compressed solid tablet or pill or as a gelatin capsule containing granules. Some patients, have, however, difficulty in swallowing such tablets or capsules. Also, it can be very difficult to persuade animals such as dogs and cats to swallow conventional dosage forms. To address this problem dosage forms that dissolve quickly on the tongue or in the mouth have been manufactured. In addition to facilitating swallowing of the dosage form when the medicine is intended for delivery into the blood stream via the gastro-intestinal tract, such so called quick-dissolving dosage forms enable delivery of medicines or drugs via the mucosa of the mouth allowing, for example, buccal, lingual or sub-lingual delivery. Delivery of drugs via the mouth mucosa should, generally, enable rapid drug delivery and is especially advantageous where the drug is intended to be delivered to the central nervous system because it enables rapid delivery of the drug to the brain and avoids or at least inhibits delivery of the drug via the gastro-intestinal tract, to non-targeted organs where the presence of the drug may have disadvantageous side effects. Also, drug absorption via the blood-rich epithelium in the mouth, rather than the chemically hostile environment of the stomach and the intestine may generally be advantageous.

Such quick-dissolving dosage forms are conventionally formed by dissolving food or pharmacological grade gelatin to form a gelatin solution containing the required active ingredient. The gelatin solution is then frozen solid converting the water content into ice and the unbound ice is then removed under conditions of low pressure causing the ice crystals to sublime. Secondary drying may also be required to remove tightly bound (sorbed) water that is strongly attached to the protein molecules. This process results in dosage forms that regularly dissolve or disintegrate in the mouth or on the tongue. However, this process is a relative complex process and generally has to be carried out as a batch-by-batch process. Moreover, gelatin is a natural product that is subject to variation in quality and solubility and certain types of gelatin may be unacceptable to certain groups of people, for example conventional animal gelatin is unacceptable to vegetarians while porcine gelatin is unacceptable to Jews and Muslims on religious grounds.

Another approach to manufacturing quick-dissolving dosage forms is described in WO 90/06969. This technique involves use of the sugar-spinning technique used for producing cotton candy or candy floss and requires the incorporation of an oleaginous substance such as vegetable oil into the sugar solution which is then melt spun in a conventional manner. This technique requires the use of melt spinnable sugars which may have disadvantages in certain circumstances. For example, the high temperature used to produce the molten sugar may have adverse effects on any active ingredient, for example, a drug to be incorporated in the dosage form.

The applicant's co-pending International Application Publication No. WO 00/67694, the whole contents of which are hereby incorporated by reference, describes another method of forming quick-dissolving dosage forms using electrohydrodynamic (EHD) processing wherein gelatin or a polymer liquid, for example a polymer solution, issuing from an outlet or nozzle is exposed to a high electric field (generated by a voltage of the order of kilovolts over a distance of some tens of centimetres) resulting in a cone and jet of liquid which dries in-flight to form a fibre which lies down onto an earthed surface to form a low-density mat of fibres having a three dimensional structure with a large surface area. This technique is suitable for batch processing as described in WO 00/67694 and may use any suitable biologically acceptable or compatible polymer that is:

(1) Suitable for electrohydrodynamic processing which generally means that the polymer is soluble in a solvent or solvent mixture that is susceptible to EHD processing such as ethanol or a similar alcohol or an ethanol or similar alcohol and water mixture; and

(2) Is soluble or will disintegrate in the environment into which the dosage form is to be placed, f or example where the dosage form is for oral delivery the polymer should be water-soluble.

It is an aim of the present invention to further facilitate the production of dosage forms using electrohydrodynamic (EHD) processing.

In one aspect, the present invention provides a method which uses electrohydrodynamic processing of artificial biologically compatible polymers to manufacture dosage forms for oral or nasal delivery of an active ingredient. Preferably, in an embodiment, the dosage forms are quick-dissolving, that is the dosage forms dissolve within 10 seconds or so or being placed in the mouth or up the nose.

A method embodying the invention may also be used to manufacture dosage forms for delivery of an active ingredient or ingredients into the ear canal or onto the cornea of an eye or for use as a vaginal or anal suppository or for, where the polymer used can be delivered into the blood stream without undesired adverse effects, for delivery of an active ingredient to the surface of a wound or opening formed by a doctor, dentist or surgeon.

In one aspect, the present invention provides a method of manufacturing a quick-dissolving dosage form, which method comprises:

supplying liquid comprising a biologically compatible polymer liquid containing particulate material that is insoluble in the polymer through a liquid supply tube to an outlet of the supply tube; establishing an electric field between the outlet and a surface spaced from the outlet to cause liquid issuing from the outlet to form a liquid jet which dries to form a polymer fibre containing particles of said particulate material and deposits onto the surface to form a tablet body consisting of said particulate containing fibre.

In one aspect the present invention provides a method of manufacturing a dosage form, which method comprises the steps of:

supplying liquid comprising a biologically compatible polymer liquid through a liquid supply tube to an outlet;

establishing an electric field between the outlet and a surface spaced from the outlet to cause liquid issuing from the outlet to form a jet which dries to form a polymer fibre which deposits onto the surface to form a first fibre layer structure;

depositing material containing an active ingredient onto the first fibre layer structure to form a region of active ingredient containing material on the first fibre layer structure; and then

forming a second fibre layer structure of a biologically compatible polymer to encapsulate said region within a fibre structure capsule formed by the first and second fibre layer structures. The first and second fibre layer structures may be formed in the same manner and using the same or different polymers.

In one aspect the present invention provides a method of manufacturing a dosage form, which method comprises the steps of:

supplying liquid comprising a solution of a biologically compatible polymer in a solvent through a liquid supply tube to an outlet;

establishing an electric field between the outlet and a surface spaced from the outlet to cause liquid issuing from the outlet to form a jet which dries to form a polymer fibre which deposits onto the surface to form a tablet body having a three dimensional fibre structure, which method further comprises:

hindering evaporation of the solvent in a region adjacent the liquid supply tube outlet where a cone and base region of a jet are formed for forming the polymer fibre. In an example, this may be achieved by controlling the partial pressure of the solvent vapour in the region of the liquid supply outlet.

The partial pressure of the solvent vapour may be controlled by locally increasing the vapour pressure of the solvent around the outlet. This may be achieved by, for example, providing a shroud or collar containing the solvent around the outlet. The solvent may comprise ethanol. In another example, the vapour pressure may be controlled by controlling the temperature at the outlet, for example by cooling the outlet to reduce evaporation of the solvent.

In one aspect the present invention provides a method of manufacturing a dosage form, which method comprises the steps of:

supplying liquid comprising a biologically compatible polymer through a liquid supply tube to an outlet; and

establishing an electric field between the outlet and a surface spaced from the outlet to cause liquid issuing from the outlet to form a jet which dries to form a polymer fibre which deposits onto the surface to form a tablet body, which method further comprises incorporating an effervescent material into the tablet body.

In each of the above aspects, the biologically compatible polymer should dissolve or disintegrate in the environment in which it is intended to be placed. At least for oral application, this means that the polymer should be water-soluble.

As used herein the terms “biologically acceptable polymer” or “biologically compatible polymer” mean any polymer that does not have a significant undesired adverse effect on the physiology of the animal or human being when the dosage form is used in the manner for which it was intended, for example when the dosage form is placed in the mouth where the dosage form is for oral use. Where the dosage form is for oral or nasal use, then suitable such polymers include PVP (polyvinylpyrrolidone) and derivatives of this polymer, for example derivatives incorporating additional co-polymers such as vinylacetate and vinylimidazole as present in Luviskol and Luvitec supplied by BASF.

Embodiments of the present invention will now be described, by way of example, with reference to the accompanying drawings, in which:

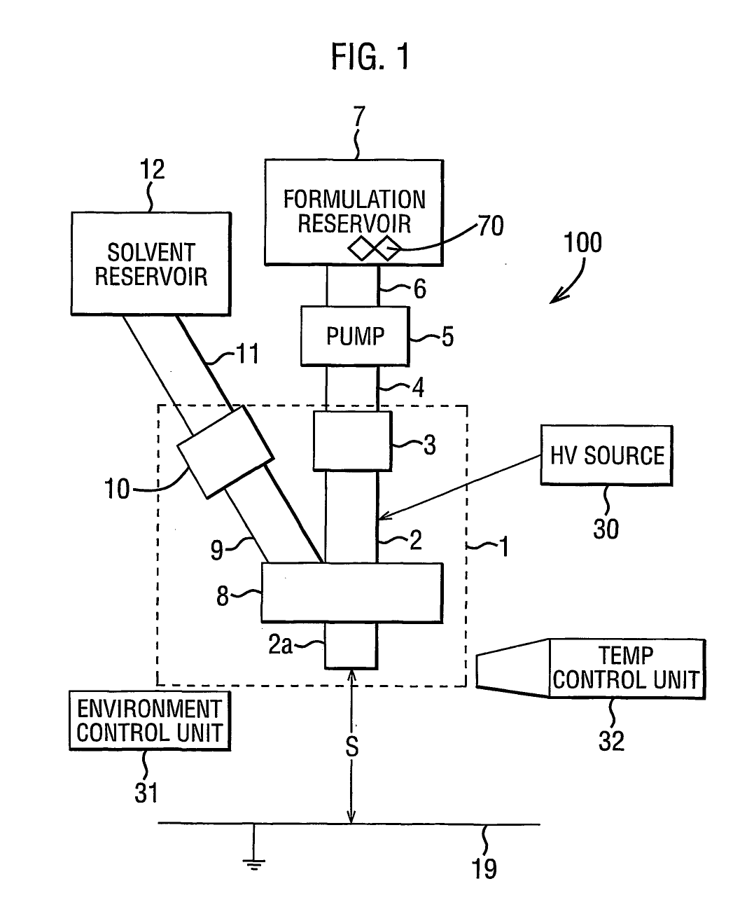

FIG. 1 shows a schematic block diagram of processing apparatus having a nozzle assembly;

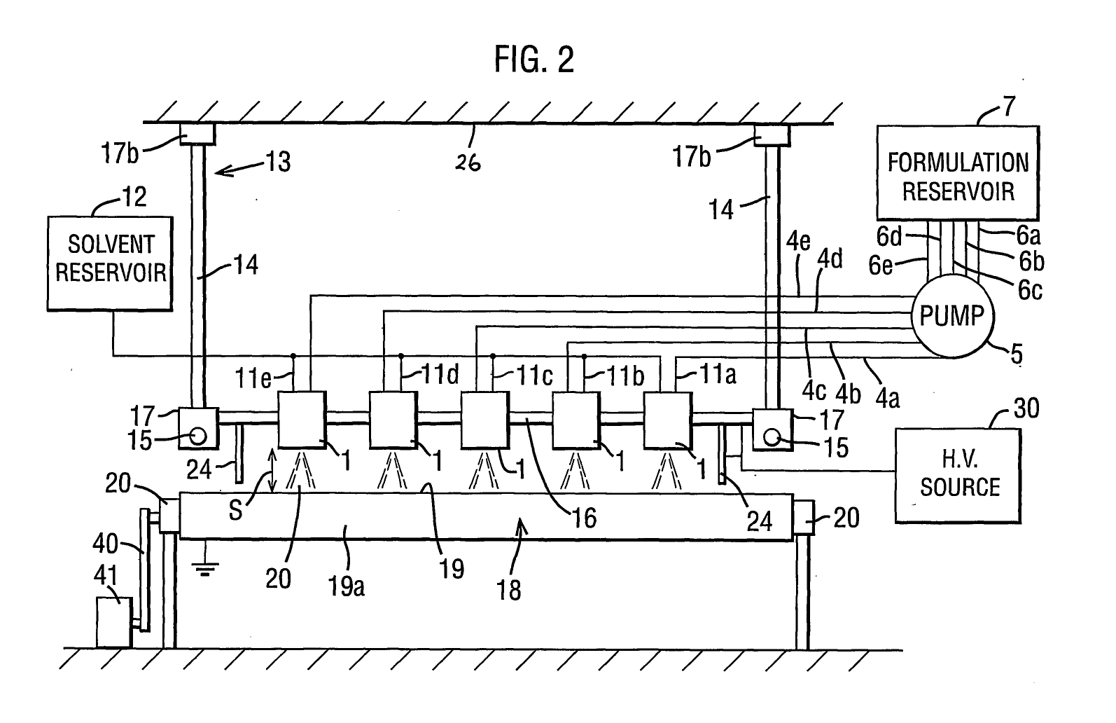

FIG. 2 shows a diagrammatic end view of a processing plant;

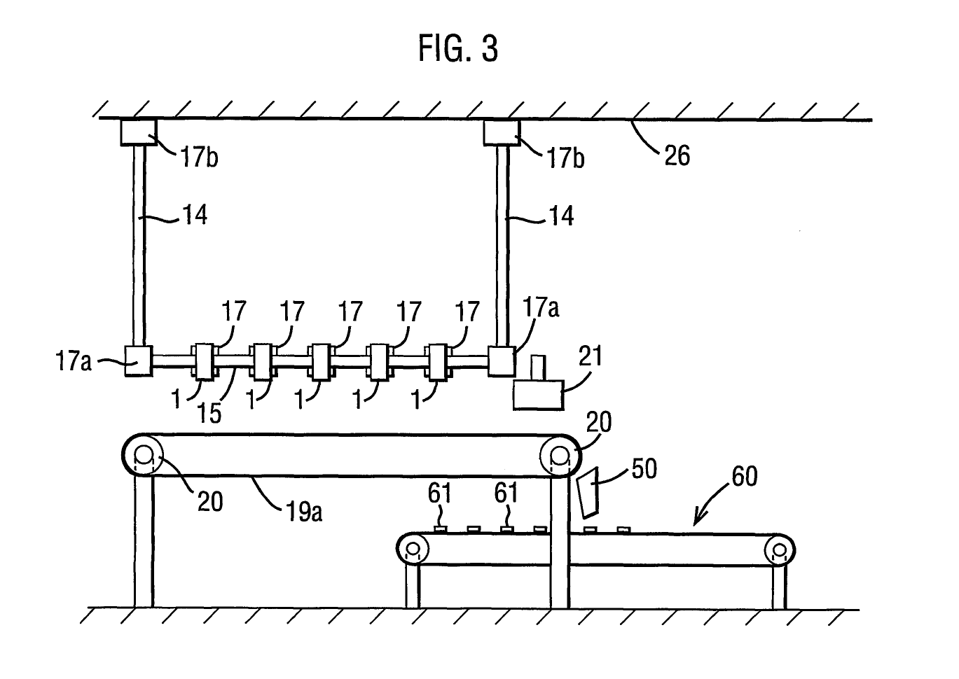

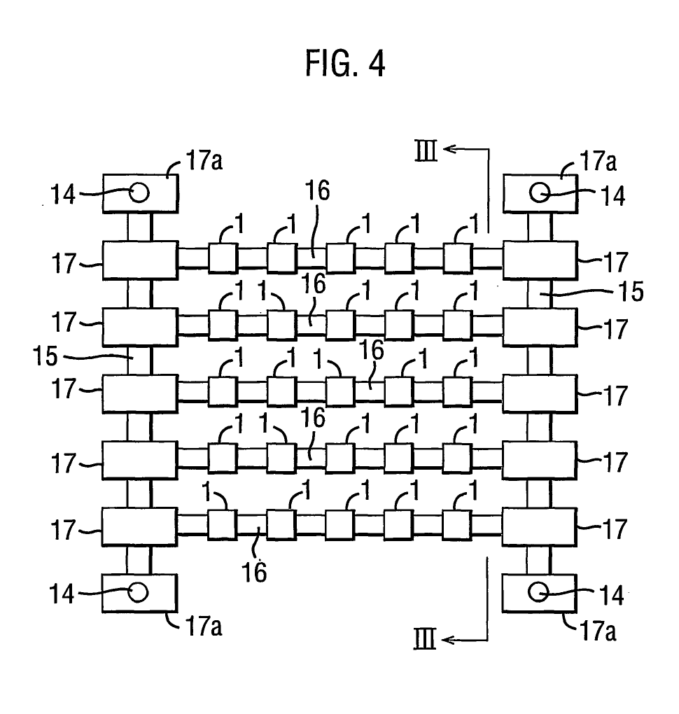

FIG. 3 shows a view, taken along line III-III in FIG. 4, of the processing plant shown in FIG. 2 with connections to the nozzle assemblies, solvent and formulation reservoirs and high voltage source omitted in the interest of clarity;

FIG. 4 shows a top plan view of a nozzle assembly support frame of the production plant shown in FIG. 2;

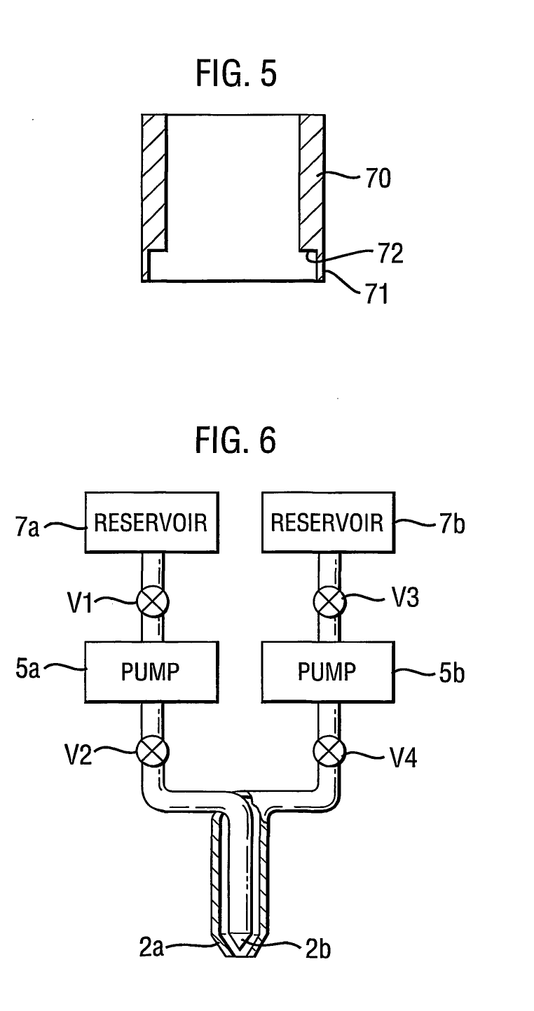

FIG. 5 shows a very diagrammatic cross sectional view though one cutting element of a cutter suitable for use in the production plant shown in FIG. 2;

FIG. 6 shows diagrammatically a modification of part of the processing apparatus shown in FIG. 1;

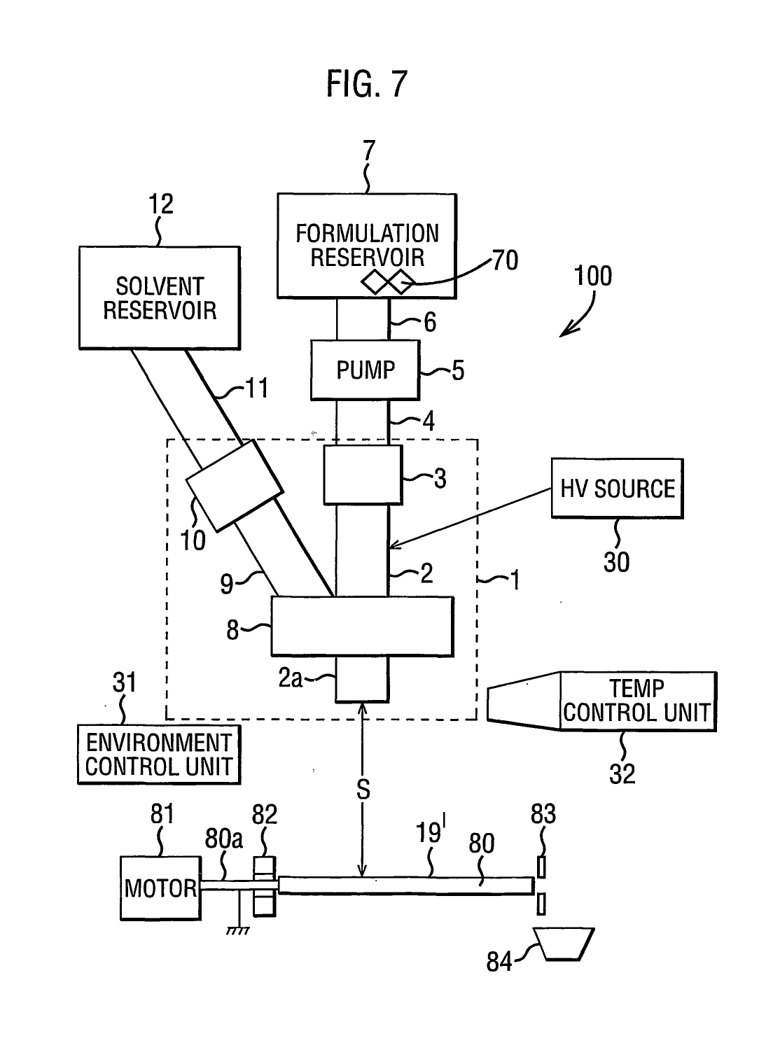

FIG. 7 shows a schematic block diagram of another processing apparatus having a nozzle assembly; and

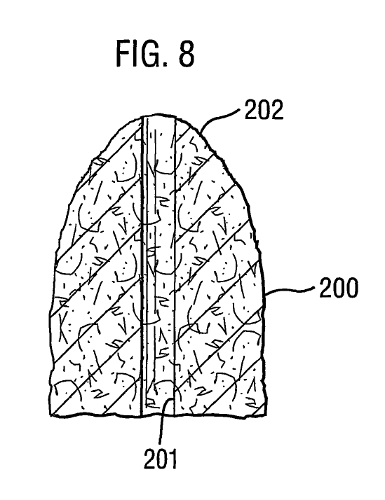

FIG. 8 shows a diagrammatic cross-sectional view of a dosage form suitable for nasal application.

Referring now to FIG. 1, a processing apparatus 100 comprises an electrohydrodynamic nozzle assembly 1 comprising a liquid supply tube 2 in the form, in this example, of an electrically conductive capillary tube having a liquid supply outlet 2a. For reasons which will be explained below, a container in the form of a shroud or collar 8 supported by and surrounding the liquid supply tube 2 may be provided and may be coupled to a supply pipe 9.

A formulation reservoir 7 for supplying to the liquid supply tube 2 a liquid solution of a biologically compatible water-soluble polymer is coupled via a supply pipe 6 to a pump 5 which is itself coupled by an electrically insulating supply pipe 4 to the liquid supply tube 2 via an electrically insulating coupling connector 3. The pump 5 may be, for example, a peristaltic pump. The formulation within the formulation reservoir may be stirred by conventional stirring means (indicated schematically in FIG. 1 by a stirring blade 70) which may be, for example, a mechanical or magnetic stirrer. It will, of course, also be appreciated that conventional arrangements will be provided for replenishing the reservoirs 7 and 12 and for inhibiting solvent evaporation from the reservoirs.

When the shroud 8 is provided, the supply pipe 9 is coupled via a coupling connector 10 to a supply pipe 11 itself coupled to the outlet of a solvent reservoir 12 arranged to supply the or a solvent of the formulation to the shroud 8 at a rate which substantially matches the evaporation of solvent from the shroud 8. The shroud 8 may contain or comprise, for example, an absorbent material such as felt to retain the solvent.

The electrically conductive liquid supply tube 2 is coupled to a high voltage source 30 arranged to provide a voltage of the order of kilovolts. Typically, the voltage supplied by the high voltage source 13 may have a value in a range of from 10 to 30 kilovolts.

The processing apparatus 100 is supported (by means not shown in FIG. 1) so that the nozzle outlet 2a is spaced by a distance or separation S above an electrically conductive support surface 19 which is coupled to earth (ground). As will be explained in greater detail below, the support surface 19 may be a movable surface such as the belt of a conventional belt drive conveyor (provided that there is an electrically conductive path to earth, for example, the belt may be electrically conductive) enabling material deposited on to the surface 19 to be moved away from the region of deposition.

In use of the processing apparatus 100 shown in FIG. 1, a biologically compatible formulation of the water-soluble polymer is pumped by the pump 5′ to the liquid supply outlet 2a and a high electric field is established between the outlet 2a and the surface 19 by the high voltage source 30. The concentration and molecular weight of the polymer are such that, as discussed in WO 00/67694, the whole contents of which are hereby incorporated by reference, a liquid cone and jet formed at the outlet 2a dries as the solvent evaporates to produce a fibre having a diameter of the order of 10 micrometres that deposits onto the surface 19 building up to form a mat or web of fibre having a relatively open three dimensional structure so that the mat or web of fibre has a high specific surface area. The belt 19 may be driven by a belt drive motor (not shown in FIG. 1) at a timing which controls the thickness of the fibre web or mat formed on the surface 19. Typically, the speed of movement of the belt 19 may be the order of few metres per minute with the actual speed depending upon, of course, the desired fibre mat thickness.

An environmental control unit 31 may be provided to control the environment in which the fibre is formed so as to control the drying of the fibre. For example, the environmental control unit 31 may control one or more of the relative humidity, temperature, air flow and partial pressure of a solvent of the polymer formulation in the environment with increase in temperature and air flow increasing the drying rate and increase in the solvent partial pressure reducing the drying rate. The support surface 19 may also be perforate (for example having the form of a porous metal mesh) to enable solvent evaporation from both sides of fibre mat.

The degree of dryness of the fibre as it deposits onto the surface 19 may also be controlled by controlling the time of flight from the outlet 2a to the surface 19 by adjusting the separation S between the outlet 2a and the surface 19. This may most easily be achieved by mounting the apparatus on a vertically movable support structure. A separate temperature control unit 32 may be provided to control the local temperature in the region of the outlet 2a. The environment in which the nozzle assemblies are placed may also be controlled to reduce the amount of, or to avoid the presence of, oxygen to minimise the risk of fire due to the evaporated solvent.

As the fibre mat or web is transported away from the location of the outlet 2a, it may be subject to further processing. This further processing may be deposition of a further material onto the fibre mat or web. This further deposition may be effected by apparatus similar to that shown in FIG. 1 located downstream of the apparatus shown in FIG. 1 to deposit onto the fibre mat or web fibres of a different polymer or to deposit particles or droplets of another material produced by EHD or another process. The fibrous mat or web resulting from any such further processing is then supplied to a conventional cutting device which divides the fibrous mat or web up into dosage forms which can then be boxed or packaged into blister packs in conventional manner, for example using a cutter similar to a pizza cutter to cut the mat or web into strips and then into dosage forms.

FIGS. 2 to 4 show very diagrammatic views of one example of a dosage form production plant constructed using apparatus similar to that shown in FIG. 1.

FIG. 2 shows an end view of the production plant while FIG. 3 shows a side view of the production plant with connections to the nozzle assemblies 1 and the formulation and solvent reservoirs 7 and 12 and high voltage source omitted while FIG. 4 shows a top plan view of a support frame or gantry carrying a nozzle assembly bank. As shown in FIGS. 2 to 4, the gantry comprises a frame 13 consisting of two longitudinal parallel spaced-apart support bars 15 and a plurality (in the example shown five) of cross bars 16, each end of which is mounted in a respective coupling block 17 slideably mounted on the corresponding support bar 15 so that the position of each cross bar 16 can be adjusted along the length of the support bars 15. Adjustment of the position of the cross bars 16 may be effected manually or under motor control in conventional manner.

Each end of each longitudinal support bar 15 is connected via a coupling block 17a (FIG. 3) similar to the coupling blocks 17 to a support upright 14 secured by a connection member 17b to a support platform or ceiling 26 of the environment within which the production plant is located. The coupling blocks 17a, like the coupling blocks 17, may enable the support bars 15 to be moved along the length of the uprights 14 to enable the height of the cross bars 16 and thus the nozzle assemblies 1 carried thereby to be adjusted. Again, this adjustment may be under manual or motor control.

Each of the cross bars 16 supports an array of nozzle assemblies 1. As shown in FIG. 2, each array consists of five nozzle assemblies although there may be fewer or more nozzle assembles.

Each nozzle assembly 1 has the structure shown in FIG. 1. As shown in FIG. 2, each nozzle assembly of an array of nozzle assemblies shares a formulation reservoir 7, solvent reservoir 12, pump 5 and high voltage source 30. Thus, the coupling elements 3 of an array of the nozzle assemblies are coupled by respective supply pipes 4a to 4e to a corresponding five tube peristaltic pump 5 whereby each of the supply pipes 4a to 4e communicates with a corresponding supply pipe 6a to 6e from the formulation reservoir 7 (FIG. 2). The coupling element 10 of each of the nozzle assemblies is coupled via a corresponding supply pipe 11a to 11e to the solvent reservoir 12 enabling solvent to be supplied to the corresponding shroud 8. The capillary tubes 2 of the nozzle assemblies 1 are coupled to the high voltage source 30 by connecting wires which, although not shown in FIG. 2, run along or through the cross bar 16. As shown in FIG. 2, the ends of the arrays of nozzle assemblies may be bounded by electrically conductive shields 24 coupled to the high voltage source 30 to reduce the possibility of tailing off of the fibre mat at the scale edges of the surface 19. As another possibility, the shields may be electrically insulating so that any fibre, ions or droplets landing on the shields do not discharge due to the electrically insulating nature of the shields but rather generate their own repulsive electric field.

Each array of nozzle assemblies supported by a cross bar 16 will have the same structure as that shown in FIG. 2. Where all of the nozzle assembly arrays are supplying the same formulation, then they may all be coupled to the same formulation and solvent reservoirs, pump and high voltage source. However, generally the different arrays of nozzle assemblies will be used to supply different material to the surface 19 and so each nozzle array will be associated with its own different formulation reservoir, solvent reservoir, pump and high voltage source. This also enables different nozzle assembly arrays to be coupled to opposite polarity high voltages. Also, although not shown, different nozzle assembly arrays may be positioned at different heights or separations S.

As shown by FIGS. 2 and 3, the gantry or frame 13 is supported above a conveyor belt assembly so that an endless belt 19a trained round rollers 20 provides the surface 19 spaced apart by the separation S from the nozzle outlets 2a. The endless belt 19a is coupled to earth and one of the rollers 20 is a drive roller coupled by, as shown in FIG. 2, a belt drive arrangement 40 to a drive motor 41. As shown in FIG. 3, a cutting device 21 is located downstream of the nozzle assembly to cut the final fibre mat or web into dosage forms of the required size. These dosage forms are then supplied, in known manner, via a hopper 50 to a further conveyor belt assembly 60 carrying peelable blister pack components 61 so that each blister pack is filled in a conventional manner to produce packs of dosage forms that allow a user to release an individual dosage form by peeling back the covering of an individual blister. As another possibility, the dosage forms issuing from the hopper 50 may be dispensed into pill or tablet bottles.

Any suitable form of cutting device may be used as described WO 00/67694.

A number of methods embodying the invention for formulating quick-dissolving dosage forms using the nozzle assembly shown in FIG. 1 will now be described.

In a first example Luvitec VI55 (a vinylpyrrolidone vinylimidazole copolymer) was used as the water soluble biological-compatible polymer. This polymer was dissolved in ethanol at a concentration of 4 grams of solid powder of the polymer to 10 ml of the solvent, in this case ethanol. The liquid supply tube 2 shown in FIG. 1 was a metal nozzle of 1.1 mm internal diameter (in this case a hypodermic needle with the chamber removed) and the pump 5 was arranged to provide a flow rate of 20 ml per hour. The nozzle outlet 2a was spaced by a separation S distance of 20 cm from the receiving surface 19 which in this example was an earthed metal plate. High voltage source 30 was arranged to provide a voltage of +20 kilovolts (kV). The environment was determined to have a humidity of 45% and the temperature was 23° C.

The formulation issuing from the outlet 2a formed a cone and a jet which jet dried inflight towards the surface 19 to produce an electrically charged fibre which deposited onto the surface 19 to form a three dimensional mat or web of fibres. The fibre mat produced by spraying (EHD processing) of 10 millilitres of polymer formulation at a flow rate of 20 millilitres per hour, that is continuous spraying for a period of half an hour, was about 10 cm in diameter and 3 mm thick. As the deposition of fibre continued, the breadth of the fibre mat formed on the surface 19 increased because the fibres already deposited insulated the newly depositing fibre from the surface 19 so that the electrically charged fibre depositing onto the surface was more readily attracted to the edge of the mat where the surface 19 was still exposed.

The above experiment was repeated varying the separation S between the outlet nozzle 2a and the surface 19 so varying the time of flight from the outlet 2a to the surface 19, and also varying the environmental temperature T into which the liquid issued from the outlet 2a.

As a result, it was found that reducing the separation S between the outlet 2a and the surface 19 (and so reducing the time of flight) resulted in a fibre mat which was more dense, less friable and less soluble in water whereas when the separation between the outlet 2a and the surface 19 was increased, the resulting fibre mat was less dense and so was more soluble but was also more friable and susceptible to separation into layers at random intervals with the layer separation frequency being dependent upon how dry the fibres were upon deposition.

It is considered that the reason for these effects is that reducing the time of flight gives fibres less chance to dry out so that the fibres are more flexible and lose their charge more rapidly because the solvent that has not yet evaporated is more conductive than the solid fibre while increasing the time of flight gives the fibres more time to dry out so that they are more rigid and remain charged for longer on the surface 19 resulting in a less dense fibre mat.

Changing the environmental temperature also affected the dryness of the deposited fibres with the dryness increasing with temperature for a given separation S.

As a result of these experiments, it was concluded that, to produce a fibre mat having similar density, the separation between the outlet 2a and the surface 19 needed to be changed by 1 to 2 cm for each ° C. change in temperature with the separation being reduced for increase in temperature and increased for reduction in temperature.

In the above described experiments, the resulting fibre had a diameter of the order of 10 micrometres with the fibres within a particular sample beings relatively monodispersed so that all the fibres had effectively the same diameter. This results in a very high surface area for a given mass of polymer making the resulting fibre mat considerable more soluble than the bulk polymer.

Experiments were also carried out varying the internal diameter of the liquid supply tube 2 or nozzle. It was, however, found that the fibre diameter was independent of this variable and that liquid supply tubes with different diameters outside the range 1 to 2 mm could also be used without affecting the fibre diameter, provided that the nozzle diameter was not so small that it restricted the supply of the formulation and not so large that gravity overcame the surface tension maintaining a continuous column of liquid in the liquid supply tube 2.

As will be understood from the above example, a relatively high concentration of polymer is required to ensure fibre formulation with a greater concentration being required for lower molecule weight than for higher molecular weight versions of the same polymer. However, problems can arise with such highly concentrated formulations in that the formulation intends to dry too quickly at the outlet 2a, forming a skin around a large stalactite type blob. The formation of such a skin can prevent the formation of the liquid cone and subsequent jet required for fibre formation or may disrupt an already-formed cone, so disrupting fibre formation. If such a skin is formed, then spraying and so fibre formation will not occur until the skin ruptures, resulting in intermittent fibre formation and a blob of semi-dry polymer at the outlet. Such blobs also have a tendency to increase in size and occasionally drop onto the already formed fibre mat, spoiling the product. Increasing the flow rate or diluting the formulation was found to compensate for this premature drying. However, increasing the flow rate or diluting the formulation results in wetter fibres which, as set out above, produce fibre mats which are denser, less friable and less soluble than before. Using a higher molecular weight of the same polymer requires a lower concentration of polymer to produce fibres and thus is less prone to premature drying. However, higher molecular weight polymers tend to be less soluble and so result in dosage forms that dissolve less quickly.

The present inventors have, however, found that the above mentioned problem of formation of large stalactite-type blobs can be controlled inhibiting evaporation of solvent in the region where the cone should form by controlling the partial pressure of a solvent of the formulation in the region of the outlet 2a. The present inventors have found that the most convenient way of controlling the partial pressure of the solvent is to locate a supply of solvent adjacent and slightly above the outlet 2a by, as described above with reference to FIG. 1, providing a solvent container in the form of the shroud or collar 8 supported on the liquid supply tube 2 adjacent the outlet 2a.

In this example, the solvent shroud 8 is formed of an absorbent material such as felt or cottonwool located about 1 to 2 mm above the outlet 2a and soaked with the solvent, in this case ethanol. Further solvent is supplied to the collar or shroud 8 from the solvent reservoir 12 via the supply pipe 11 at a flow rate designed to replace the solvent as it evaporates. If necessary, the absorbent material forming the shroud 8 may be supported within an insulative material mesh-like housing.

The partial pressure of the solvent or its evaporation rate may be adjusted by heating or cooling the absorbent material forming the collar or shroud 8 using the temperature control unit 32 which may be in the form of, for example, an air blower controllable to blow either cold or hot air onto the shroud 8.

Thus such skin formation can be avoided by providing the shroud 8 and supplying solvent from the reservoir 12 to the shroud 8 to increase locally the partial pressure of the solvent in the region where the cone is formed. This increase in partial pressure of the solvent inhibits solvent evaporation and enables reliable fibre formation with high polymer concentration polymer formulations. This facilitates the use of low molecular weight polymers (which require high polymer concentration in the formulation to produce fibres) which is particularly advantageous because low molecular weight polymer fibres tend to dissolve more rapidly than high molecular weight polymer fibres.

As described above, the problems of premature drying out of the formulation at the outlet 2a can be avoided by controlling the partial pressure of a solvent of the formulation at the outlet 2a. Other ways of inhibiting solvent evaporation in the region of the cone may be used, for example local temperature control may be used to lower the temperature at the outlet, so reducing evaporation.

The above described experiments have been repeated with other polymers. In particular, in addition to Luvitec, PVP (polyvinylpyrrolidone) and other PVP derivatives such as Luviskol VA55E (which is a vinylpyrrolidone/vinylacetate copolymer manufactured by BASF of 67056 Ludwigshafen Germany) have been used.

Thus, in another example, a Luviskol VA55E formulation having a concentration of 5 grams of polymer in 10 ml of ethanol was used as the formulation and, under the same processing conditions as given above for the Luvitec formulation, produced a fibre mat having coarser fibres that were stronger but less friable and less soluble than the fibres produced using the Luvitec formulation.

Further experiments were carried out using as the polymer formulation a formulation consisting of a combination of the above Luviskol and Luvitec formulations. Combining the two formulations resulted in a fibre mat with properties in between those of the fibre mat formed using Luvitec alone and Luviskol alone. Increasing the amount of Luvitec in the formulation increased the solubility and friability of the resultant fibre while increasing the amount of Luviskol reduced the solubility and the friability of the resultant fibre mat. Fibre mats with good solubility that are not too friable (which can make the fibre mat too delicate to be handled) where obtained using a 1:1 mixture of Luviksol:Luvitec.

Similar processing conditions have been used to produce fibre mats using PVP as the water soluble biologically compabitable polymer. For example, a PVP concentration of 6 grams of PVP of molecular weight 40,000 in 10 ml of ethanol or a concentration of 1.5 g in 10 ml of ethanol for PVP of molecular weight 360,000 have been used. The fibre mats produced using PVP tend, however, to be less soluble than the fibre mats produced using Luviskol or Luvitec with fibre mats producing using PVP of molecular weight 40,000 being rather skin-like, friable and less soluble and fibre mats produced using PVP of molecular weight 360,000 being less dense than the fibre mats produced using PVP of 40,000 molecular weight but still less soluble than the fibres mats produced using Luvitec or Luviskol.

The present inventors have, however, found that the characteristics of the resultant fibre mat or web can be improved so that the fibre matrix is less dense and more soluble by incorporating in suspension in the polymer formulation particles of a material that is insoluble in the polymer formulation. It is thought that the particles assist in allowing a greater height or distance S (and so a longer drying time) by possibly increasing the inertia and may also reduce the required drying time by reducing the proportion of solvent in the formulation and fluffing up the fibres on the deposition surface, so allowing greater drying on the surface.

The particulate material may be formed of any one or more of a number of different types of materials with the only constraint being that the particulate material is biologically compatible and is substantially insoluble in and can be suspended in the polymer formulation. Examples of types of particulate material are: inert materials (that is materials that have no significant biological, chemical or physiological effect on the user when the dosage form is used in the manner intended) such as chalk or kaolin particles or particles of another biologically compatible polymer that is insoluble in the polymer formulation; where the dosage form is for oral delivery, flavourings such as sweeteners both artificial and natural (such as simple and complex sugars) and/or saliva stimulants and/or effervescent particles, that is particles that effervesce in the mouth but not in the polymer formulation; particles of active ingredient. All of these types of particles may be solid, hollow or porous. Suspending hollow particles in the formulation used to produce the fibres so that the hollow particles are incorporated into the fibre or depositing hollow particles onto the fibre in flight or after deposition should increase the solubility of the tablets and the particles themselves should dissolve or disintegrate more readily than solid particles. Other types of particles that may be used include: micro capsules (formed of, for example, another biologically compatible polymer that is insoluble in the polymer formulation); that are inert (i.e. contain air, gas or an inert liquid) or contain an active ingredient or ingredients in solid, granular, liquid or gel form; polymer particles having dissolved or dispersed therein an active ingredient; particles of active ingredient coated with a coating material that is insoluble in the polymer formulation such as another biologically compatible polymer. Any one or more of these types of particles may be used and, where the particles are or incorporate an active ingredient, one or more different active ingredients may be used, depending upon the properties required of the dosage form. The particles may be of the same size (mono-dispersed) or may have a range of sizes, being, when generally spherical, smaller than 1 mm in diameter and typically having a diameter or diameters in the range from sub micron to 100 microns. The particles need not necessarily be spherical but could be ellipsoidal, granular, shard-like or rod-like, for example.

Surprisingly, the present inventors have found that it is not necessary for the particulate size to be smaller than that of the fibres. Rather similar effects have been obtained with particulates having a particle size or a distribution of particle sizes in the range from sub micron to greater than a 100 micrometres (the larger size particles tend to be aggregations of smaller size particles). The suspended particles are trapped within the resultant fibres so that even when their diameters are many times greater than the diameter of the fibre the particles have a polymer coating.

The size of the particulate (particles or agglomerations of particles) affects the resultant product. The larger the particles or agglomerates are the fluffier/more rigid/more brittle the fibre mat tends to be.

The actual concentration of particulates in the formulation can be varied from, for example, 0.1 to 1.0 grams per ml of polymer formulation where the particulate material is provided to improve the characteristics of the fibre mat or web and may form up to 80% by weight of the final product dosage form where the particulate material comprises an active ingredient, dependent on the required dosage of active ingredient.

In one experiment, a polymer formulation consisting of grams of PVP of molecular weight 40,000 and 0.1 grams of PVP of molecular weight 360,000 per 10 ml of ethanol was modified by adding particulate material in the form of 2 grams of icing sugar per 10 ml of polymer formulation. This modified formulation was supplied through the liquid tube 2 (with an internal diameter of 1 mm) at a flow rate of 20 ml per hour. The environmental temperature was again 23° C., the separation S was 30 cm, the voltage applied by the high voltage source 30 again +20 kilovolts and spraying or fibre production continuing for 20 minutes. As a result, a fibre mat having an elliptical area of roughly 16 by 19 cm with a depth of 2.3 mm and a density of 0.145 grams per cm³ was formed. The fibres were white and the fibre mat had a non-brittle, non-friable nature. Circular pills having a diameter of 15 mm and a mass of 63 mg were cut or punched from this fibre mat. These fully dissolved after 10 seconds in tap water at 18° C.

A further experiment was carried out using the same formulation and the same processing conditions apart from the fact that the separation S was reduced to 20 cm so that the fibres had less drying time. In this case, the resultant fibre mat consisted of an elliptical area of 10×11 cm of white fibres with a central glassy region having an area of approximately 3×4 cm. The glassy region had a plastic nature and a density of about 1 gram/cm³ suggesting that it was an amorphous solid. The surrounding area had a depth of 1.5 mm, a density of 0.39 grams/cm³ and was very brittle. Circular pills of 15 mm in diameter and a mass of 111 mg cut from this fibre mat took approximately 40 seconds to fully dissolve in tap water at 18° C.

As can be seen by comparing the two sets of results, the change in the separation S significantly affected the characteristics of the fibre mat. However, although the appearance of the fibre mat resulting when the separation S was reduced was less desirable and the resultant product was more difficult to handle because of the brittle nature of the surrounding area, the circular pills produced from this fibre mat still fully dissolved in tap water relatively quickly.

As set out above, the time of flight of the fibres influences the density of the resultant fibre mat and, in the case where the separation S was reduced to 20 cm, the glassy region in the fibre mat was directly beneath the outlet 2a and so thus resulted from fibres having a shorter time of flight than the fibres in the peripheral area. Indeed, in this case, the fibres in the glassy region was so damp that they completely merged together. As set out above, the formation of such a glassy region can be avoided by, when using a separation S of 20 cm, increasing the environmental temperature.

Further experiments were carried out using as the polymer formulation a formulation having 2 grams of PVP of molecular weight 40,000 and 1 gram of PVP of molecular weight 360,000 in 10 ml of ethanol with varying amounts of icing sugar in suspension in the polymer formulation. In these examples, a liquid supply tube in the form of a delrin capped metal nozzle of internal diameter 1 mm was used, the voltage applied by the high voltage source 30 was +20 kilovolts, the surface 19 was an earthed metal surface, the separation S was 26 cm, the environmental temperature was 22° C., the relative humidity was 24% and fibre formation was continued for 20 minutes.

In a first experiment, no particulate material was suspended in the polymer formulation. In this case, the resultant fibre mat had an elliptical area of approximately 13×16 cm with a depth of 1 mm and a density of 0.212 grams/cm³ and was formed of dense white fibres. Circular pills of 15 mm diameter and a mass of approximately 4 mg were cut from this fibre mat. These pills were found to be very brittle and very soluble in water at 18° C.

In a second experiment, 1 gram of icing sugar was added per 10 ml of the above polymer formulation to produce a particulate suspension. The resulting fibre mat had an elliptical area of approximately 15×17 cm with a depth of 1.2 mm and a density of 0.177 grams/cm³ and was formed of dense white fibres. An approximate fibre density of 0.132 grams/cm³ was estimated by compensating for the mass of the particulate material (icing sugar) by reducing the mass of the fibre mat by 1 quarter. Again, 15 mm diameter pills of approximately 40 mg mass were cut from the fibre mat. These pills were less brittle than the pills produced using no icing sugar but were still very soluble.

In a third experiment, the above formulation was modified by adding 5 grams of icing sugar per 10 ml of the above polymer formulation, again producing a particulate suspension. In this case, the resulting fibre mat had an elliptical area of approximately 21×24 cm with a depth of 2.2 mm and a density of 0.96 grams/cm³ and was formed again of dense white fibres. An approximate fibre density of 0.036 grams/cm³ was estimated by compensating for the mass of icing sugar by reducing the mass of the fibre mat by ⅝. Again, 15 mm diameter pills of approximately 40 mg mass were cut from the fibre mat. These pills were even less brittle, although slightly more friable (this could be compensated for by reducing the separation S slightly) and again very soluble.

Although the pills produced in each of the three experiments were of similar solubility in tap water it is important to note that the less dense pills float better and so have fewer fibres exposed to the tap water at any one time. Taking this into account, the less dense pills should dissolve more quickly in the mouth, that is the pills incorporating the icing sugar should dissolve more quickly than those without the icing sugar. Further experiments have been carried out using the above three formulations but continuing spraying of fibre production for longer (for example, up to one hour) so producing thicker fibre mats and therefore resulting in thicker pills. In these cases, the unloaded pills (that is the pills containing no icing sugar) proved to be less soluble than the pills containing the icing sugar.

The fact that the influence of the icing sugar was not due to the presence of minute quantities of icing sugar dissolved into the formulation was confirmed by allowing a suspension to settle out and then spraying the remaining polymer formulation (containing any dissolved icing sugar) under the same conditions as set out above. The resulting fibre mat was comparable to that obtained using the formulation that did not contain any icing sugar.

Further experiments were carried out using different water soluble biologically compatible polymers. In one experiment, two polymer formulations were produced (one formulation containing as the polymer PVA of molecular weight 49,000 and the other formulation containing as the polymer PVA of molecular weight 130,000) each consisting of 1 gram of polymer in 10 ml of solvent consisting of a 1:1 mixture of water and ethanol. Two batches of each formulation were produced and particles of amylose suspended in one batch of each formulation at a concentration of ˜3 grams of amylose in 10 ml polymer formulation. The processing conditions were as set out below:

Formulation containing PVA of molecular weight 49,000

Flow rate 10 ml per hour

Separation S 16 cm

Voltage Applied+30 kV

Formulation containing PVA of molecular weight 130,000

Flow rate 10 ml per hour

Separation S 16 cm

Voltage Applied+20 kV

As in the case of the PVP formulations described above, the unloaded formulations (that is the formulations not containing the amylose suspension) produced fibre mats having a central glassy region while the loaded formulations (that is the formulations containing the amylose suspension) produced fibre mats having of similar surface area to those produced with the unloaded formulation but having a greater depth (less than 1 mm to between 1 and 2 mm deep) indicating that the fibre mats produced using the loaded formulations were less dense. The fibre mats resulting from use of the loaded formulations were also more robust and easily handled and so were easily separated from the surface 19.

A further experiment was carried out using as the polymer polycaprolactone of molecular weight 65,000 at a concentration of 0.2 grams per ml of acetone and with icing sugar suspended in the formulation at a concentration of 3 grams of icing sugar per 10 ml of formulation. Although the icing sugar particles tended to settle out in the liquid supply tube (because of the lower density and lower viscosity of the formulation) what particles that did get through into the fibres appeared to make the product less dense.

Similar results have been obtained by incorporating as the particulate material glass beads having a diameter of 35 microns to provide a formulation consisting of 3 g of glass beads plus 5g of PVP of molecular weight 40,000 (PVP₄₀ₖ) and 0.1 g of PVP of molecular weight 360,000 (PVP₃₆₀ₖ) in 10 ml of ethanol and by incorporating as the particulates 2 micron PTFE (polytetrafluoroethylene) spheres to provide a formulation consisting of 2 g of mono-disperse 2 micron PTFE spheres suspended in 10 ml of polymer formulation consisting of 5 grams of pVP₄₀ₖ plus 0.1 grams of PVP₃₆₀ₖ per 10 ml of ethanol. Of course, such beads would not generally be used in an ingestible tablet. Rather these experiments serve to show that, because similar results were obtained using chemically inert glass or PTFE beads as the particulate, the effect of the particulate is not a chemical effect and therefore similar effects should be seen with almost any particulate matter that is not soluble or significantly soluble in (or does not react with) the polymer formulation.

The introduction of the particulates appears to increase the rigidity of the fibres and thus reduces the density of the resulting fibre mat. Increasing the concentration of particulates increases the brittleness and friability of the fibres and accordingly the concentration of particulates can be used to control the degree of brittleness or friability of the resultant fibre mat.

In the above examples, the maximum concentration of particulates that can be incorporated into suspension in the polymer formulation depends upon the polymer and the present inventors have found that the amount of particulate material than can be suspended within a polymer formulation before fibril (short fibre lengths) formulation occurs can be increased by using a higher molecular weight of the same polymer with the structural effects of the suspended particles more than compensating for the reduced solubility of the polymer. Thus, for example, a polymer formulation containing 2 grams of PVP of 40,000 molecular weight and 1 gram of PVP of 360,000 molecular weight in 10 ml of ethanol can support 6 grams of particulate material in suspension and still produce a robust pill that disperses quickly in water.

Where the formulation reservoir has a mechanical or magnetic stirrer or agitator, this may be used to maintain the suspension. Where the particulates are of particularly high density, they may, however, settle out in the supply pipe 4 resulting in an inhomogeneous mixture. The possibility of settling out can be reduced by using smaller diameter tubing to make the formulation move more quickly along the tube and also by shaping the tube to facilitate mixing within the tube, for example, by causing the tube to bend or zigzag to ensure mixing of top and bottom layers of the formulation at each corner. Other methods of agitating the suspension to avoid settling of the particulate material may be used such as, for example, ultrasound.

It can be seen from the above described experiments that the presence of particulates in suspension in the polymer formulation greatly reduces the density of the resulting fibre mat and also generally improves its physical characteristics (solubility in water and robustness in handling). The incorporation of particulates in suspension in the polymer formulation may be used in combination with the control of the partial pressure of the solvent described above to control the characteristics of the resultant fibre mat or web.

Further increasing the concentration of particulates may cause the fibres to break up into fibrils (short lengths of fibre) before reaching the surface 19. This can be used to deliberately produce short lengths of fibre. This may be used, for example, to enable one of the banks of nozzles shown in FIGS. 2 to 4 to produce fibrils (which may contain an active ingredient) for incorporation into a fibre mat being produced by adjacent banks of nozzle assembles.

As described above, fibrils containing particulate material such as an active ingredient may be produced by one or more of the nozzle assemblies discussed above.

Additionally or alternatively one or more of the nozzle assemblies may be used to produce polymer coated particles that deposit onto the fibre mat being formed by other nozzle assemblies by controlling the concentration of polymer in the formulation so that the viscosity is insufficient to suppress the perturbation of the jet that occurs during EHD processing and the jet breaks up into droplets which consist of active ingredient particles coated by polymer.

The polymer coating may have one or more functions depending upon the nature of the polymer and the intended use of the dosage form. For example, where the dosage form is for oral delivery, the polymer coating may be a polymer such as ethyl cellulose that is substantially insoluble in the mouth so providing a physical barrier that prevents the user tasting the actual active ingredient which may have an unpleasant taste. Additionally or alternatively, the polymer coating may enable controlled or targeted delivery of the active ingredient, for example the polymer coating may dissolve only slowly in the mouth (it could even be the same polymer as the fibre mat but sufficiently thick that it takes a relatively long time to dissolve to expose the active ingredient) or may remain intact until the coated particle reaches a lower part of the gastrointestinal tract where the polymer may be dissolve or degrade under enzymic or chemical attack. For example, ethyl cellulose will dissolve in the acidic environment of the stomach.

In any of the above examples, dispersion or solubility of the pills manufactured from the fibre mat in water can be aided by the incorporation in the polymer formulation of an effervescent agent that is biologically compatible (for example a mixture of citric acid and sodium hydrogen carbonate) which evolves gas on contact with water but not in the ethanol based polymer formulation. This evolution of gas agitates the water around the pill increasing its rate of dispersion of the fibre matrix and the affect is also rather pleasant to the patient. A particular advantage of the use of electrohydrodynamic processing to produce dissolving pills is that the effervescent can be incorporated into the pill. In contrast, conventional methods for producing quick-dissolving pills generally require the use of water as part of their formulation or production process which makes the incorporation of an effervescent impossible because water is, of course, the catalyst for the effervescent reaction.

Quick-dissolving dosage forms produced using the method described above will, as mentioned above, generally incorporate an active ingredient, that is a biologically compatible ingredient that has a therapeutic or otherwise desirable effect on the consumer of the pills. For example, the active ingredient may be a biologically active chemical entity for the prevention or cure of disease or alleviation of symptoms such as a drug, a medicament, food supplement, biological material such as DNA, DNA fragments, a protein and so on.

As described above, active ingredient may be suspended (possibly coated or encapsulated) in the polymer formulation. Other or additional techniques for incorporating one or more active ingredients may be used as described below.

Where the active ingredient is soluble within the polymer formulation, then the active ingredient may be fully dissolved within the polymer formulation. Although dissolving of an active ingredient into the polymer formulation is convenient and enables the density of active ingredient in the resulting dosage forms to be controlled relatively easily, the chemical nature of the active ingredient can affect both the EHD processing of the formulation (that is the ability of the formulation to produce fibres) and also the properties of the resultant fibre mat or product. For example, incorporation of 0.2 grams per ml of aspirin, paracetamol or an ibuprofen sodium salt into the PVP formulations described above increases the viscosity of the formulations significantly. Indeed, such a concentration of paracetamol or ibuprofen disrupts the fibre production almost completely. This concentration of aspirin within the polymer formulation allows the production of suitable fibres but the resulting fibre mat was dense and less soluble than the fibre mat without the active ingredient and took longer to dry than the fibre mat without the active ingredient. Moreover, because of the hygroscopic nature of the active ingredient, the fibre mat actually absorbed moisture from atmosphere making the product pliant and less soluble.

Although not all active ingredients that are soluble within the polymer formulation may have such deleterious effects, it is important to find a way to enable medicaments such as these popular over-the-counter medicaments to be incorporated as the active ingredient.

Where controlled or relatively slow release is required, then this type of active ingredient may be coated or encapsulated as described above. The present inventors have found that another way of incorporating such soluble active ingredients into the quick-dissolving pill is to spray material containing the active ingredient onto the fibre mat as it is being formed. This may be achieved by, for example, using some of the nozzle assembly arrays shown in FIGS. 2 to 4 to produce fibres for forming the fibre mat others to produce droplets containing the active ingredients.

The formulation used to produce the droplets containing the active ingredient may be a lower concentration formulation of the same polymer with the amount of polymer in the formulation being so low that fibre production does not occur, rather liquid droplets are formed which are sprayed by the nozzle assembly array directly onto the fibre mat being produced. The nozzle assembly array or arrays producing the active ingredient containing droplets may have a high voltage source 30 of the same or the opposite polarity. Where provided, such droplet spraying nozzle assemblies should be evenly distributed between fibre spraying apparatus to produce an even distribution of droplets throughout the mat or web being formed.

Charging the active ingredient containing droplets to the opposite plurality makes the resultant fibre mat denser and less friable.

As another possibility droplets containing the active ingredient may be sprayed onto the fibres in-flight by directing the outlet of a droplet producing nozzle assembly towards a fibre producing nozzle assembly. Indeed spraying from the droplet producing nozzle assembly may be induced by the charged fibre being produced by the fibre producing nozzle assembly. In an example, the droplet producing nozzle was positioned 26 cm above the deposition surface 4 cm away from and 1 cm lower than the fibre producing nozzle. In this case, the droplet producing nozzle was earthed while a voltage of +20 kv was applied to the fibre producing nozzle. The fibre formulation consisted of a suspension of icing sugar in polymer formulation with a concentration of 2 grams of icing sugar per 10 ml of polymer formulation and a polymer formulation consisting of 5 grams of PVP₄₀ₖ plus 0.1 grams of PVP₃₆₀ₖ per 10 ml of ethanol while the droplet formulation consisted of 1 gram of ibuprofen, 1 ml of ethanol and 4 ml of corn oil. During the EHD processing, the droplets produced by the droplet producing nozzle were mainly attracted to and thinly coated the fibres. In-flight droplet spraying has the advantage of producing more even distribution of droplets within the resulting fibre mat but the disadvantage of causing at least some electrical discharging of the fibres reducing their ability to settle on the earthed surface 19. The present inventors have, however, found that this electrical discharging of the droplets can be controlled by, for example, ensuring that the formulation used to produce the droplets is more resistive than the formulation used to produce the fibres (so that it holds less charge) and by controlling the respective flow rates and voltages used. Another way of controlling undesired electrical discharging of the fibre in-flight is to ensure that the droplets remain as discrete entities on the fibre. Generally, the total droplet charge should be of the order of one tenth of the fibre charge. Also the droplets may be electrically discharged by using a discharge electrode as described in, for example, U.S. Pat. No. 4,962,885. Supplying the active ingredient in such a droplet formulation has the advantage that the active ingredient does not affect the fibre formation.

The formulation used to produce the droplets containing the active ingredient need not necessarily contain the same polymer as the formulation used to produce the fibres and can be chosen for compatibility with the active ingredient. The droplets may be gel-like or liquid and still tacky when the fibre deposits on the fibre mat.

Techniques other than electrohydrodynamic processing may be used to spray active ingredient onto a fibre as it is being produced or onto the fibre mat as it is being produced, for example a conventional liquid droplet or aerosol production process or a triboelectric process may be used.

As described above, where the active ingredient is insoluble in the polymer formulation being used to produce the fibres, then the active ingredient may be incorporated within the polymer formulation as a particulate suspension. The amount of particulate that can be suspended within the polymer formulation can be increased by using a higher molecular weight polymer with the reduced solubility of the polymer being more than compensated for by the structural effects of the suspended particles. As a higher molecular weight polymer requires a lower concentration to produce fibres than a lower molecular weight of the same polymer, this also has the advantage of increasing the ratio of active ingredient to polymer within the resulting fibre mat, allowing production of dosage forms having a high concentration of active ingredient.

An active ingredient, whether soluble or insoluble within the polymer formulation for producing the fibres, may be pre-processed by encapsulation or coating within a different, secondary material that is immiscible with the fibre forming polymer formulation using electrohydrodynamic processing to produce solid droplets or to produce fibrils containing the active ingredient (where the concentration of particulate material within the polymer formulation is, as described above, sufficient to cause resulting fibre to break up into fibrils). The secondary material may be a polymer, or as another possibility a lipid-based material or wax. Such encapsulated active ingredient particles may be manufactured in advance and mixed into the fibre formulation to form a suspension or may be manufactured in situ by either mounting the nozzle assembly for forming the active ingredient containing solid droplets or fibrils above the formulation reservoir 7 so that the droplets or fibrils are sprayed directly into the formulation reservoir 7 and providing a stirrer or other agitation mechanism within the formulation reservoir 7 to ensure uniform dispersion of the encapsulated or coated active ingredient or by spraying the droplets or fibrils onto the fibre and/or fibre mat during its formation.

Encapsulating or coating the active ingredient in a secondary material, for example a second polymer, has advantages in addition to inhibiting any deleterious effect of the active ingredient on the fibre formulation or resultant fibre mat. Thus, for example, the secondary polymer selected for forming the droplets or fibrils encapsulating the active ingredient may be a biologically compatible polymer that is not or is only slightly water soluble but that dissolves, disintegrates or degrades when subject to chemical or enzymic attack, for example, within the stomach or another part of the gastrointestinal tract. An example of a polymer that does not dissolve in the PH neutral environment of the mouth but will dissolve in the acidic environment of the stomach is ethyl cellulose and the inventors have produced droplets of ethyl cellulose containing aspirin using a formulation containing 0.2 grams per ml of aspirin within a 0.15 gram per ml ethyl cellulose (5-15 cps, in a standard solution) in ethanol solution.

Rather than being encapsulated, the active ingredient may be dissolved or suspended within a secondary material such as a polymer that is not dissolvable within the environment of the mouth. Where the active ingredient is suspended, then the secondary material will generally completely surround or encapsulate most of the particles in a manner similar to that described above for fibre production incorporating particulate matter. However, when the active ingredient is dissolved within the secondary material, the active ingredient and secondary material form a dense particle in such a way that water can only slowly penetrate the active ingredient-secondary material matrix and hence can only slowly dissolve the active ingredient. Slow release of dissolved active ingredient from a droplet or fibril that is not dissolvable in PH neutral water has been demonstrated by replacing the active ingredient by a coloured food dye. In this case, when the droplets are suspended in PH neutral water, about half the dye was released after 20 minutes. Even slower release of the dye (and so of the active ingredient) may be achieved by using a higher concentration of polymer so that there is a greater proportion of polymer in the droplets while avoiding fibre formation (possibly by incorporation of chemically inert particulates to break the fibre up into fibrils).

Another way in which fibre formation could be avoided in these circumstances is to use a lower molecular weight (lower viscosity) ethyl cellulose which has a similar solubility in water but which can be used in higher concentration in the droplet formulation before it produces fibres.

Where the dosage form is for oral delivery, incorporation or encapsulation of the active ingredient into a polymer droplet or fibril that does not dissolve or degrade or disintegrate in the environment of the mouth also has the advantage that, because no or very little active ingredient is released within the mouth, taste of the active ingredient should be masked or at least reduced. Thus, this polymer coating or encapsulation acts as a physical taste barrier preventing the consumer from tasting or reducing the degree to which the consumer can taste the active ingredient which may be particularly advantageous in the case of a drug such as ibuprofen which has a particularly unpleasant taste.

To illustrate these effects, polymer droplets consisting of ibuprofen particles coated by ethyl cellulose were produced by EHD processing using a separation S of 26 cm, a flow rate of 10 millilitres per hour of formulation (consisting of 0.4 grams of the pharmaceutically acceptable acid version of ibuprofen (that is ibuprofen USP) dissolved in a polymer formulation consisting of 0.15 g of ethyl cellulose per millilitre of ethanol) and deposited onto a layer of PVP fibre produced by EHD processing for a duration of 60 seconds at a flow rate of 20 millilitres per hour with a separation S of 26 cm and a voltage of +/−20 Kilo volts (kV)). These steps were repeated ten times to complete the fibre mat, ending with a fibre layer. The resulting fibre mat was divided into dosage forms of 15 mm diameter, 2 mm depth. These had a mass of approximately 50 mg and, on the basis of spectrophotometer results, contained 14 mg of ibuprofen. When tasted, the unpleasant taste of ibuprofen was much less noticable than with dosage forms in which the ibuprofen particles were not coated. Similar effects were achieved with up to 10 grams of ibuprofen in the droplet formulation.

An example will now be described in which pills or tablets containing a taste-masked active ingredient mimic were produced. In this example, two sets of the apparatus shown in FIG. 1 were provided and were positioned relative to a moving circular track so as to spray EHD produced matter onto opposite edges of the moving circular track. The formulation reservoir 7 of one of the sets of apparatus contained a fibre producing formulation while the formulation reservoir of the other set of apparatus contained a droplet producing formulation. In this example, the fibre producing formulation consisted of a polymer formulation with 4 grams of amylose suspended per 20 ml of polymer formulation with the polymer formulation comprising 5 grams of PVP of molecular weight 40 k and 0.1 grams of PVP of molecular weight 360 k per 10 ml of ethanol. In the droplet formulation reservoir, 4 grams of icing sugar were suspended per 10 ml of droplet formulation with the droplet formulation consisting of 1.5 grams of ethylcellulose per 10 ml of ethanol. The flow rate for the fibre formulation was 20 ml per hour while the flow rate for the droplet formulation was 10 ml per hour. In each case, the high voltage source 30 provided a voltage of +30 kv. As EHD processing continued alternate layers of fibres and droplets built up on the moving circular track. The resulting fibre mat was divided up into rectangular pills. When the pills were tasted the presence of the icing sugar could not be detected, indicating that the encapsulation of the icing sugar particles in the ethylcellulose had masked the taste of the icing sugar.

Another way in which active ingredient can be incorporated into the quick-dissolving dosage forms is to deposit the active ingredient as a mound onto a fibre layer to form an active ingredient region. In this case, once the active ingredient region has been formed, then a second fibre layer structure is formed using a further bank of nozzle arrays as described above and dosage forms cut out from the resulting fibre mat using a cutter which seals the edges of the dosage form to form a flying saucer or pillow-like shape. A diagrammatic example of a single cutting element (for producing a single pill) of such a cutter is shown in FIG. 5. As can be seen from FIG. 5, this cutting element 70 has a peripheral sharp or knife edge 71 for cutting through the fibre mat to form the dosage form and an inner sealing edge 72 for sealing together the cut edge of the first and second fibre layers. A heating element may be incorporated into the cutter to heat the sealing edge 72 so that thermal sealing is effected

The first and second fibre layer structure may be formed of the same or opposite polarities. Where the fibre layer structures are formed of opposite polarity this may be sufficient to seal the edges. As another possibility the EHD processing conditions used to produce the second layer of fibres may, following the teaching above, be controlled so that the fibres are initially slightly damp and so bond to the first layer of fibres.

A dispersion agent, such as an effervescent (for example a mixture of citric acid and sodium hydrogen carbonate) can be incorporated within the active ingredient region along with conventional taste masking and flavouring components where necessary so that the experience of consuming the dosage form is not unpleasant). Also the active ingredient “powder” may be at last partially polymer coated active ingredient with the polymer coating being only slowly soluble or insoluble in the mouth, thereby providing a taste barrier as described above.

Such a sandwich-like structure may be also be used where the active ingredient is to be a liquid or is dissolved in a liquid that is poorly volatile because the sponge like nature of the fibre matrix will retain the active ingredient prior to consumption.

Another example of taste masking in such a sandwich-like structure will now be described. In this case, coated taste-masked ibuprofen was isolated from a Nurofen Meltlet tablet by disintegrating a tablet in distilled water, filtering off the solids, washing them a few times in distilled water and then drying. The resulting of agglomeration was gently broken up and about 100 milligrams of sherbert was added for taste/effervescence to about 260 milligrams of the agglomeration. In this case the fibre formulation consisted of 2 grams of particulate material (in this example icing sugar) in 10 ml of polymer formulation (5 grams of PVP 40K plus 0.1 grams PVP 360K in 10 ml of ethanol). Processing was continued to produce a fibre mat sandwich-like structure with the thickness of about 6 mm (millimetres) and this was divided into square pills having sides of 25 mm.

As will be appreciated from the above, active ingredient encapsulated in or coated by a second polymer or distributed throughout a secondary polymer matrix may, depending upon the second polymer, be incorporated into the fibre forming polymer solution, may be sprayed onto the fibre or the fibre mat or web as it is being produced or may be provided as a mound or pile in a sandwich-like fibre structure. Two or more different polymers may be used which have different disintegration or dissolving characteristics. Thus, the same dosage form may contain droplets or fibrils of different polymers containing the same or different active ingredients. As an example, the polymer droplets or fibrils may include polymer droplets or fibrils of ethyl cellulose and also of a polymer that does not dissolve or degrade until it reaches the intestine such as cellulose acetate phthalate (CAP) or cellulose acetate hydrogen phthalate (CAHP). In this case, when the dosage form is placed within the mouth, the fibre matrix will dissolve or disintegrate readily in the mouth but both the ethyl cellulose and CAP polymer droplets will remain substantially intact and will be swallowed by the consumer. The ethyl cellulose polymer droplets will be degraded or dissolved within the acidic environment of the stomach so releasing their active ingredient while the CAP polymer droplets will remain intact and will not disintegrate or dissolve until they reach the alkaline environment of the small intestine. This enables controlled release of the same drug or delivery of different drugs to different targeted areas. Of course, the same dosage form may also incorporate an active ingredient in the fibres of the fibre mat so that this active ingredient is delivered primarily to the environment of the mouth, for example by buccal, sub-lingual or lingual delivery. A single dosage form may thus enable delivery to any one or more of such targeted areas.

Such encapsulated or coated active ingredient may be produced by EHD processing as described above or by other, conventional means.

As shown in FIG. 6 and as described in WO 00/67694, the nozzle or liquid supply tube of the electrohydrodynamic processing apparatus used to produce the polymer droplets fibres or fibrils may be modified to enable production of multilayer fibres, droplets or fibrils. Thus, as shown in FIG. 6, the single liquid supply tube shown in FIG. 1 is replaced by concentric first and second liquid supply tubes 2a and 2b each connected via a respective valve V2 and V4 to a respective pump 5a and 5b itself connected by a respective valve V1 and V3 to a respective formulation reservoir 7a and 7b. (Although not shown a solvent shroud 8 may be provided adjacent the concentric outlets 2a and 2b).

Each of the reservoirs 7a and 7b contains a liquid formulation containing a polymer. In this example, the reservoir 7b contains a liquid formulation containing ethyl cellulose as the polymer and the other reservoir 7a contains a liquid formulation containing amylose as the polymer. This concentric arrangement allows one jet of formulation to pass through the centre of the other so that when the jet issuing from the concentric outlets 2a and 2b forms a fibre, breaks up into fribils or forms polymer droplets, the fibre, fribil or droplet has an inner core of one polymer and an outer coating of the other polymer. When such fibres, fibrils or droplets are incorporated into a dosage form then the outer polymer, in this case ethyl cellulose, will provide a protective barrier so that the inner polymer core is not exposed until the outer ethyl cellulose coating has been degraded or disintegrated in the acidic environment of the stomach.

Another method of obtaining such core or coated droplets is to spray droplets of the core polymer formulation onto a surface and then suspend them in the coating polymer formulation or to spray them directly into the coating polymer formulation.

The tablets or fibre mats discussed above may be encapsulated in an outer coating which will dissolve or disintegrate during the intended use of the tablet. For example, tablets intended for oral use may have an outer sugar coating produced by depositing sugar onto the tablets and then fusing. As another possibility, a tablet may be encapsulated in gelatin or inserted into a pre-formed gelatin capsule. As another possibility, a support surface, for example formed of rice paper where the tablet is intended to be ingested, may be provided on the deposition surface to assist in removal of the tablet from the support service and to assist the tablets in maintaining integrity during handling. As a further possibility, the EHD processing characteristics may be changed so that the outer fibre effectively forms a coating or casing having a larger diameter than the inner fibre and generally containing no active ingredient.

The surface onto which the fibre is deposited may be perforate or apertured to enable drying from both sides.

Flavouring may be added to the tablet not only to assist in masking or reducing the taste of active ingredient but also to, for example, stimulate the appetite or to stimulate saliva production to promote the dispersion of the tablet.

Use of PVP and PVP derivatives such as Luviskol and Luvitec is particularly advantageous because they are both water soluble and soluble in the solvents that are particularly suitable for electrohydrodynamic processing such as ethanol. However, any biologically compatible polymer that is water soluble and soluble in the solvent to be used for EHD processing may be used.

Polymers suitable for fibre, droplet and controlled delivery via EHD processing are shown in the following table:

| POLYMER | SUPPLIER |
|---|---|
| Polylactic acid | |
| Polyglycolic acid | |
| Luvitec | BASF |
| Luviskol | BASF |
| Polycaprolactone | |
| Polyethyleneglycol (PEG) | |
| PEG_polyester | |
| Polyphosphate esters | |
| Polyorthoesters | |
| Polyanhydrides | |
| Artificial proteins | |
| Polycarbonates | |
| Polyiminocarbonates | |
| Polyarylates | |
| Polyphosazenes | |
| Gelatin and Derivatives | |
| Cellulose acetate | |
| phthalate | |
| Chitin | |
| Plasdone | ISP-Pharma |
| Povidone | ISP-Pharma |
| Copolyvidonum | ISP-Pharma |
| Polyplasdone | ISP-Pharma |
| Crospovidone | ISP-Pharma |
| Eudragit | Röhm |
| Polysucrose | |
| Amylose and Derivatives | |
| Cellulose | |
| Biocompatible ceramics | |
| Polymers with Active | |
| Ingredients chemically | |
| bound to them | |

In the above described examples, the dosage form is designed to dissolve or disintegrate quickly. However, the polymer fibre mat may be designed to adhere to a surface against which it is placed (for example to a surface within the mouth by forming an outer layer of the dosage form of a mucosal adhesive or incorporating a mucosal adhesive in the dosage form, examples of mucosal adhesives being given in, for example, WO 94/20070 and polycyanoacrylates) and to dissolve relatively slowly, possibly turning to a gel, by, for example, making some of the fibre thicker or of a less water soluble polymer, releasing its active ingredient locally (eg via the mouth mucosa when placed in the mouth) and over a period of time.

Such a dosage form may adhere to, for example, the sub-lingual or buccal area of the mouth so enabling delivery of the active ingredient over a period of time for absorption through the sub-lingual or buccal surface without any significant ingestion of the active ingredient.

The present inventors have found that such a dosage form can be produced by modifying the Luviskol/Luvitec formulation given above by using a version of Luviskol with a lower proportion of the acetate copolymer (40% instead of 50%). Surprisingly, this results in a dosage form that does not disperse rapidly in water but rather forms a gel which may adhere to a surface such as buccal or sub-lingual surface in the mouth. Adhesion may be facilitated by inclusion of mucosal-adhesives and electret polymers within the formulation or by providing an outer coating of a mucosal adhesive as described above.

The use of synthetic polymers as opposed to the gelatin that is normally used to produce quick-dissolving dosage forms has a number of advantages. In particular, such polymers can, unlike gelatin, be purely water soluble.

Furthermore, gelatin is a natural product so that it has a variable quality and variable solvation and EHD processing characteristics making it rather unreliable for production processes. In addition, gelatin is an animal product and so is not suitable for vegetarians and may be objectionable on religious grounds (for example porcine gelatin will be objection to Jews and Muslims).

In the above embodiments, evaporation of solvent in the cone region (the cone and base of the jet) is controlled to inhibit evaporation. In some circumstances it may however, be desirable, where the formulation is particularly wet, to enhance evaporation using a desiccant in the container 8. In the above described examples, evaporation of the solvent is relied upon to cause drying of the jet to form a fibre. Other techniques may be used to dry the fibres such as, for example, curing by electromagnetic radiation such as UV curing where the polymer is so susceptible or reaction action with a component in the atmosphere into which the jet issues.

As described above, the dosage forms are intended for oral delivery. The above described techniques may also be used to produce dosage forms for nasal delivery because the resultant dosage forms require very little moisture to cause them to become gel-like. For example, the dosage forms may be designed and shaped to be inserted into a nasal passage up to just before the turbinates. This should enable rapid delivery of active ingredient via the nasal mucosa with the advantage of a much more accurate control over dosage than can be achieved with the use of an inhaler. A discussion of transport of drugs from the nasal cavity to the central nervous system can be found in a paper entitled “Transport of drugs from the nasal cavity to the central nervous system” by Lisbeth Illum published in the European Journal of Pharmaceutical Sciences volume 11, 2000 pages 1 to 18.

In the above described embodiments, the surface 19 is a movable belt. FIG. 7 shows a diagrammatic representation similar to FIG. 1 of processing apparatus designed to enable production of dosage forms for nasal delivery.

In FIG. 7, the support surface 19′ is provided by a mandrel 80 that is mounted on a shaft 80a that is rotatable by a motor 81 and that is coupled to earth.

In use of the processing apparatus 100′ shown in FIG. 7, the motor 81 is activated to rotate the mandrel 80 and fibre produced by the EHD processing deposits onto the surface 19′ building up to form a fibre network or body having a relatively open three dimensional structure so that the mat or web of fibre has a high specific surface area. In this case, a fibre body is built up which, because the mandrel 80 is rotating, has the form of a generally cylindrical hollow body. The thickness of the cylinder wall of the fibre body is controlled by controlling the time for which the fibre is deposited. Typically, the mandrel will have a diameter of and the deposition will be continued so that the fibre body has a diameter that is typically in the range one half to 1 cm depending upon the average size of the nasal passage of the intended end user. The mandrel may be hollow to enable temperature control (by blowing hot or cold air through the mandrel) and may have a perforate wall to facilitate drying as discussed above.

After deposition of the desired thickness of fibre onto the mandrel 80 to form the fibre body, the resulting fibre body is divided along its length to form a number of cylindrical dosage forms each of which typically is 1 to 2 cm in length. FIG. 7 illustrates very diagrammatically one way in which the fibre body may be provided into dosage form. Thus, in this example, a pushing device in the form of a collar 82 is slidably mounted on the shaft 80a and, once the desired thickness of fibre body has been formed on the mandrel 80, is moved axially along the mandrel 80 to push the fibre body of the mandrel 80 between reciprocating cutter blades 83 of a cutting device that slices the fibre body radially to define the individual dosage forms which then are collected in a hopper 84. The blades of the cutting device may simply cut the fibre body up into small hollow cylindrical dosage forms. The cutting device may comprise two pairs of spaced apart cutting blades one of which defines a rounded front end and the other of which defines a flat rear end for the dosage form. As another possibility, a single pair of cutting blades may be provided that provide the dosage form with a rounded insertion end and a correspondingly recessed rear end. As another possibility, the cutting device may be supplemented by an abrading tool which rounds off or slightly compresses the insertion end of the dosage form to facilitate its insertion.

The individual dosage forms may then be packaged into bottles or blister packs in conventional manner.

FIG. 8 shows a cross sectional view through an example of a dosage form 200 suitable for nasal delivery having an axial through hole 201 defined by the mandrel 80 and a rounded insertion end 202. The axial through hole 201 serves to enable a user to breath freely when the dosage form is first inserted into a nasal passage.

The dosage form may be provided with a thin polymer coating to facilitate handling.

In use, after removal of the dosage form from its bottle or blister pack, the user grabs the dosage form 200 between the thumb and forefinger and then inserts it gently into one nostril. The user may then use a finger, thumb or small pencil-like insertion device to push the dosage form slightly into the nasal passage so that, for example, the insertion end 202 of the dosage form is just below the turbinates. The through-hole 201 enables the user to breath freely when the dosage form is first inserted and the environment within the nasal passage causes the dosage form to dissolve or disintegrate as the user inhales, thereby delivering the medicament carried by the dosage form to, for example, the blood stream via the nasal passage.

In the example described with reference to FIG. 7, the fibre is deposited onto a rotating mandrel. As another possibility, the surface 19′ that receives the fibre may be, for example, the surface of a conveyor belt which moves at a speed sufficient to enable a thickness of fibre equivalent to the desired length of the dosage form to be built up and then transferred to a dosage form defining station at which the fibrous mat is received on a first cutting surface carrying retractable cylindrical punches which cooperate with an opposed cutting surface carrying cylindrical cutters so that, when the two cutting surfaces close together and the cylindrical punches are extended, cylindrical fibre bodies are cut from the fibre mat, each having an axial through hole defined by the cylindrical punch. After retraction of the cutting device carrying the circular cutters, the dosage form, supported on the cylindrical punches may be transported to an abrading or finishing tool that rounds off the exposed end of each fibre body to produce a final dosage form having the structure shown in FIG. 8.

As another possibility, fibre may be deposited into a hollow tube (possibly using an air flow to draw the fibre into the tube) so that the fibre deposits on the inside of the tube to form a generally cylindrical body which can then be gently pushed out of the tube and sliced transversely of its length to form tablets or dosage forms.

In the above described examples, the dosage forms for nasal application have an axial through-hole to enable a user to breath freely when the dosage form is first inserted into a nasal passage. The provision of such a through-hole may, however, not be necessary, because the dosage form structure should be quite porous. As another possibility, more than one breathing passage way may be provided through the dosage form.

A mucus production stimulant may be added to the dosage form to assist in dissolving or disintegrating of the dosage form.

In another example, dosage forms for nasal application are produced with a skin-like outer surface or coating, typically 50 to 100 micrometres thick, and formed of a gel like material such as gelatin or a water-soluble or bio-resorbable polymer. This skin-like coating may contain granular or particulate material having a particle or granule size sufficient to inhibit passage into the wind pipe. Such a dosage form may be formed by producing a skin-like material layer, for example using electrohydrodynamic processing, of gelatin or a water-soluble or bio-resorbable polymer, then using a suction technique to draw the skin-like layer into moulds defining the desired shape of the dosage forms, for example generally bullet-shape, to define a receptacle or casing for the granular or particulate material. The granular or particulate material, carrying one or more active ingredients, may then be loaded into these casings from a discharge nozzle adapted to discharge predefined quantities of the particulate or granular material as the mould carrying the skin-like receptacles is moved in a conventional indexed manner beneath the discharge nozzle. Once the casings have been loaded with the granular or particulate material, a further skin-like layer may be deposited over the casings and heat sealed to the casings to produce the dosage forms. These may then be packaged in conventional manner in, for example, blister packs. The granular (approximately 1 mm particles) material may be formed by crushing or grinding up the above described dosage forms.

The above described techniques for manufacturing dosage forms for nasal application may also be used to manufacture oral dosage forms.

As another possibility, the dosage may be inhaled in a granulated form (with the dosage form being crumbled by the user or granulated by the manufacturer before packaging). Such a nasal suppository may be used for many different drugs and has the advantage that higher doses can be achieved than when using a nasal spray or inhaler.

The dosage forms described above may also be designed for insertion into the ear canal, for use as anal or vaginal suppositories and for placement in the eye (for example on the cornea) or in a tooth cavity or for local drug delivery to treat or alleviate sore throats, mouth ulcers etc. They may also be used on wounds, if the polymer or polymers used can be introduced into the bloodstream without significant adverse effects.

In the above embodiments, the surface 19 or 19′ is earthed which has the advantage that nozzle assemblies can use different polarity high voltage sources. However, the surface 19 or 19′ may alternatively be held at high voltage.

As used herein the term “active ingredient” includes any substance that has an effect, generally not an adverse effect, on the human or animal body when the dosage form is used in the manner intended, examples are drugs, medicaments, food supplements, prophylactics, confectionary products, breath freshners, placebos and biological molecules including for example DNA, DNA fragments, proteins and such like.

The production of dosage forms designed for nasal delivery may be particularly advantageous for delivery of peptides, hormones and proteins such as insulin, calcitonin and growth hormones. These are chemically and biologically unstable which means that they are frequently administered using subcutaneous or intramuscular injections as well as intravenous infusions. The short biological half-lives of these drugs, usually in the range of several minutes, requires in some cases frequent injections and may cause considerable discomfort to patients, especially where long term or chronic treatment is necessary. Incorporation of such active ingredients into a dosage form for nasal delivery should avoid these problems and also help avoid liver first-pass effects.

In addition, dosage forms designed for nasal delivery may be particularly advantageous for paediatric immunisation programs which currently include a large number of injections in the first few months of life. Nasal vaccine delivery would eliminate the need for needles. Nasal vaccine delivery may also induce immunity at the site of infection. For example, a potential treatment of respiratory syncytial virus (RSV) disease is immunoprophylaxis. RSV-enriched immunoglobulin (RSVIG) reduces the severity of RSV infection when given prophylactically to high-risk patients, such as children under age two with bronchopulmonary dysplasia, or premature babies. Unfortunately, RSVIG only gives temporary immunity to patients, and therefore needs to be given every month during the RSV season. Nasal delivery of these active ingredients should enable such treatment without the need for too many injections.

In addition, DNA or RNA vaccines can induce potent humoral and cellular immune responses; however, these vaccines have been administered parentally. More effective protection against mucosal pathogen could be achieved with mucosal immunisations, and therefore delivery of genes to the nasal epithelium have vast potential in prevention or treatment of many diseases, such as allergic diseases, cystic fibrosis and lung cancers.

As used herein the term “flavouring” includes any substance that improves or desirably changes or affects the taste of the dosage form, when the dosage form is used in the manner intended. Examples are sweeteners such as simple and complex sugars and artificial sweeteners and other materials (for example orange or lemon flavours) commonly used in the pharmaceutical industry to hide or improve the taste of drugs or medicaments to be taken by mouth.

Although polymer solutions are discussed above, at least for some polymers it may also be possible to provide the polymer formulation as a melt.

As will be understood from the above, a dosage form embodying the invention may be any solid dosage form formed from a three-dimensional fibre network as described above. The word solid is intended here to indicate that the dosage form is in, primarily, the solid rather than liquid phase and there are, of course, interstices in the fibre network. The dosage form may be designed as: tablet or pill to be taken by mouth or to be placed in the mouth; to be inserted into a nasal passage; to be inserted into an ear canal; to be placed on an eye; as a vaginal or anal suppository; or where the polymer forming the dosage form has no significant adverse effect if it enters the bloodstream on the surface of a wound or opening or cavity formed during surgery including dental work. The dosage form may, because of the way in which it is manufactured, have, in plan, any desired shape suitable for its intended use. For example, dosage forms for oral delivery may be round, square, polygonal (for example octagonal), star-shape and so on and may carry printing or be coloured to identify the dosage form or active ingredient.

1. A method of manufacturing a dosage form, which method comprises the steps of:
providing a liquid comprising a biologically compatible polymer formulation containing a suspension of particulate material that is insoluble in the polymer formulation;
supplying the liquid to a liquid supply tube having an outlet;
establishing an electrical field between the outlet and a surface spaced from the outlet to cause liquid issuing from the outlet to form a jet which dries to form a polymer fibre containing particles of the particulate material and which deposits onto the surface to form a dosage form body consisting of said particulate containing fibre which fibre dissolves or disintegrates in a wet environment such as the mouth.

2. A method according to claim 1, wherein the particulate material comprises inert particles.

3. A method according to claim 1 or 2, wherein the particulate material comprises a flavouring agent.

4. A method according to any one of the preceding claims, wherein the particulate material comprises particles of another polymer.

5. A method according to any one of the preceding claims, wherein the particulate material comprises particles of an active ingredient.

6. A method according to any one of the preceding claims, wherein the particulate material comprises an active ingredient coated or encapsulated in another a polymer that is substantially insoluble in said polymer.

7. A method according to any one of the preceding claims, wherein the particulate material comprises polymer particles having active ingredient dispersed therein.

8. A method according to any one of the preceding claims, wherein the particles have a size or sizes in a range of from submicron to up to or greater than 100 microns.

9. A method according to any one of the preceding claims, which comprises providing the polymer formulation as a polymer solution and such that the concentration of particulate material in suspension is at least comparable to the concentration of polymer in the solution.

10. A method according to any one of the preceding claims, which comprises providing the polymer formulation such that the dosage form is about 80% by weight of particulate active ingredient.

11. A method according to any one of the preceding claims, wherein the polymer formulation is an ethanol solution of at least one of PVP and a PVP derivative.

12. A method according to any one of claims 1 to 8, wherein the polymer formulation comprises 2 grams of PVP of molecular weight 40,000 and 1 gram of PVP of molecular weight 360,000 in solution in 10 ml of ethanol and between 1 and 10 grams of particulate material per 10 ml of formulation.

13. A method according to any one of claims 1 to 8, wherein the polymer formulation is a solution of 5 grams of PVP of 40,000 molecular weight and 0.1 grams of PVP of 360,000 molecular weight in 10 ml of ethanol and 2 grams of particulate material per 10 ml of formulation.

14. A method according to any one of claims 1 to 8, wherein the polymer formulation comprises a polymer solution comprising a mixture of PVP of molecular weight 40,000: PVP of molecular weight 360,000 in the range of 50:1 to 2:1 with between 1 and 10 grammes of particulate material, with the amount of particulate material increasing with the proportion of PVP of molecular weight 360,000.

15. A method according to any one of the preceding claims, comprising incorporating in the fibre mat particles of a different polymer containing an active ingredient, said different polymer being dissolvable or degradable in part of the gastro-intestinal tract but not in the mouth.

16. A method according to claim 15, wherein said different polymer is ethyl cellulose.

17. A method according to claim 15, wherein said different polymer encapsulates a region of a further polymer containing active ingredient, whereby, when the dosage form is consumed, the different polymer dissolves or disintegrates in one part of the gastro-intestinal tract and the further polymer is dissolved or disintegrated in another part of the gastro-intestinal tract.

18. A method according to claim 15, 16 or 17, which comprises providing said polymer particles by encapsulating active ingredient within said different polymer.

19. A method according to claim 15, 16 or 17, which comprises providing the polymer particles so that active ingredient is distributed throughout said different polymer.

20. A method according to any one of the preceding claims, which further comprises incorporating an effervescent material into the fibre dosage form.

21. A method according to claim 20, wherein at least some of said particulate material comprises said effervescent material.

22. A method according to any one of the preceding claims, which comprises forming a first layer structure of fibres, depositing material comprising an active ingredient onto the first fibre layer structure to form an active ingredient region, forming a second fibre layer structure on top of the active ingredient region so that edges of the first and second layer structures seal together to define the dosage form.

23. A method according to claim 22, which comprises incorporating an effervescent material into the active ingredient region.

24. A method according to any one of the preceding claims, which further comprises controlling evaporation of a solvent from a cone region formed at the outlet of the liquid supply tube.

25. A method according to claim 24, which comprises controlling evaporation by controlling the partial pressure of the solvent in the cone region.

26. A method according to any one of the preceding claims, which comprises controlling the environment in which the fibre is formed to control drying of the fibre.

27. A method according to any one of the preceding claims, which further comprises dividing the tablet body into a plurality of tablets.

28. A method of manufacturing a dosage form, which method comprises the steps of:
supplying a liquid comprising a biological compatible polymer to a liquid supply tube having an outlet;
establishing an electrical field between the outlet and a surface spaced from the outlet to cause liquid ejection from the outlet to form a jet which dries to form a polymer fibre which deposits onto the surface to form a first fibre layer structure which dissolves or disintegrates in a wet environment such as the mouth;
supplying material containing an active ingredient onto the first fibre layer structure to form a region of active ingredient containing material on the first fibre layer structure;
supplying liquid comprising a biologically compatible polymer to a liquid supply tube having an outlet;
establishing an electric field between the outlet and the surface spaced from the outlet to cause liquid issuing from the outlet to form a jet which dries to form a polymer fibre and deposits to form on the active ingredient region a second fibre layer structure which dissolves or disintegrates in a wet environment such as the mouth; and
causing the first and second fibre layer structures to join together around said region of active ingredient to form a tablet in which the active ingredient region is encapsulated within a fibre capsule formed by the first and second fibre layer structures.

29. A method according to claim 28, which further comprises providing at least one of a flavouring and an effervescent material inside the fibre capsule.

30. A method according to claim 28 or 29, which comprises forming the first and second layer structures of the same polymer.

31. A method according to any one of claims 28 to 30, which comprises providing at least one active ingredient within the biologically compatible polymer liquid.

32. A method according to any one of claims 28 to 31, which comprises providing particles in suspension in the biologically compatible polymer liquid.

33. A method according to claim 32, wherein the particles comprise inert particles.

34. A method according to claim 32 or 33, wherein the particles comprise particles of another polymer.

35. A method according to claim 32, 33 or 34, wherein the particles comprise particles of active ingredient.

36. A method according to claim 32, 33, 34 or 35, wherein the particles comprise at least one of active ingredient coated or encapsulated in polymer or polymer particles having distributed therein active ingredient.

37. A method according to any one of claims 28 to 36, wherein the polymer is provided in solution and the method further comprises controlling evaporation of a solvent of a solvent of the solution in the region of the outlet.

38. A method of manufacturing a dosage form, which method comprises the steps of:
providing liquid comprising a solution of a biologically compatible polymer in a solvent;
supplying the liquid to a liquid supply tube having an outlet;
establishing an electrical field between the outlet and a surface spaced from the outlet to cause liquid issuing from the outlet to form a cone and jet which dries to form a polymer fibre and which deposits onto the surface to form a tablet body which will dissolve or disintegrate in a wet environment such as the mouth, which method further comprises:
controlling solvent evaporation in the region of the cone.

39. A method according to claim 38, which comprises controlling solvent evaporation by controlling the partial pressure of the solvent in the region of the liquid supply outlet.

40. A method according to claim 39, wherein the step of controlling the partial pressure comprises increasing the solvent partial pressure in the vicinity of the liquid supply outlet.

41. A method according to claim 40, which method comprises controlling the partial pressure of the solvent by providing a solvent-containing shroud around the outlet.

42. A method according to any one of claims 38 to 41, which method comprises suspending particulate material within the liquid.

43. A method according to claim 42, wherein the particulate material comprises particles of at least one of:
an inert material;
a flavouring;
another polymer;
an active ingredient;
a polymer encapsulated or coated active ingredient;
a polymer having active ingredient distributed therein.

44. A method according to any one of claims 38 to 43, further comprising incorporating into the dosage form active ingredient coated by encapsulated or distributed in another polymer that is not dissolvable or degradable in the mouth but dissolves or degrades in part of the gastro-intestinal tract.

45. A method according to claim 43 or 44, wherein said other polymer is ethyl cellulose.

46. A method according to any one of claims 38 to 45, which further comprises incorporating an effervescent in the dosage form.

47. A method according to claim 46, which comprises incorporating the effervescent as particulate material suspended within the polymer liquid.

48. A method according to any one of claims 38 to 47, which comprises sandwiching a region of active ingredient between first and second layers of fibres.

49. A method of manufacturing a dosage form, which method comprises the steps of:
providing a liquid comprising a biologically compatible polymer;
supplying the liquid to a liquid supply tube having an outlet;
establishing an electrical field between the outlet and a surface spaced from the outlet to cause liquid issuing from the outlet to form a jet which dries to form a polymer fibre that deposits onto the surface to form a dosage form which will dissolve or disintegrate in a wet environment such as the mouth, which method further comprises:
incorporating an effervescent material into the dosage form.

50. A method according to claim 49, wherein the step of incorporating the effervescent material comprises incorporating the effervescent material as a suspension of particles within the liquid.

51. A method according to claim 50, wherein the step of incorporating the effervescent material comprises sandwiching the effervescent material between layers of fibres.

52. A method according to claim 49, 50 or 51, wherein the polymer liquid is a polymer solution and the method further comprises controlling solvent partial pressure in the vicinity of the outlet.

53. A method according to claim 52, which comprises controlling the solvent partial pressure by providing a shroud or collar containing solvent around the outlet.

54. A method of preparing an active ingredient for a dosage form, which method comprises the steps of:
providing a liquid comprising a biologically compatible polymer containing a suspension of particulate active material that is insoluble in the polymer;
supplying the liquid to a liquid supply tube having an outlet;
establishing an electrical field between the outlet and a surface spaced from the outlet to cause liquid issuing from the outlet to form a jet which breaks up to form polymer droplets containing particles of the particulate active material, the polymer being insoluble or not very soluble in a wet environment such as the mouth so that the polymer provides a barrier against a user tasting the active ingredient.

55. A method according to claim 54, which comprises providing a coating of another polymer on the polymer droplets.

56. A method according to claim 54, wherein said polymer is dissolvable or degradable in the stomach but not in the mouth.

57. A method according to claim 56, wherein said polymer is ethyl cellulose.

58. A method according to claim 54, which comprises providing a polymer coating on said polymer, whereby when the polymer droplet is consumed, the different polymer dissolves or disintegrates in the stomach and the polymer is dissolved or disintegrated in the intestine.

59. A method of manufacturing an oral dosage form which comprises using a method in accordance with any one of the preceding claims to form the dosage form.

60. A method of manufacturing a dosage form to be inserted into a nasal passage, which method comprises using a method in accordance with any one of claims 1 to 58 to form the dosage form.

61. A method according to claim 60, which further comprises providing the dosage form with at least one through-hole or aperture.

62. A method of manufacturing a dosage form, which comprises using a method in accordance with any one of claims 1 to 58, which comprises providing the fibre such that it dissolves or disintegrates in the wet environment to provide a gel-like body that adheres to a surface of the wet environment, for example a surface of the mouth cavity.

63. A method according to claim 62, which comprises providing a mucosal adhesive in or on the dosage form.

64. A dosage form comprising a body consisting of a three dimensional network of a biologically compatible polymer fibre which dissolves or disintegrates in a wet environment such as the mouth, the polymer fibre containing particles of a particulate material that is insoluble in the polymer.

65. A dosage form comprising a region comprising at least one of an active ingredient and an effervescent encapsulated within a casing formed by a three dimensional fibre mat or web formed by a biologically compatible polymer which dissolves or disintegrates in a wet environment such as the mouth.

66. A dosage form according to claim 64 or 65, wherein the fibre contains active ingredient.

67. A dosage form according to claim 65 or 66, wherein the actual ingredient is encapsulated or distributed within a different polymer that does not dissolve in the mouth but will dissolve or degrade in part of the gastro-intestinal tract.

68. A dosage form according to claim 67, wherein said different polymer comprises ethyl cellulose.

69. A dosage form according to claim 67 or 68, wherein said different polymer provides a coating around a core of a further polymer encapsulating or having distributed therein an active ingredient, said further polymer being adapted to dissolve or disintegrate in a different, lower part of the gastro-intestinal tract than said different polymer.

70. A dosage form according to any one of claims 64 to 69, wherein the fibre forming polymer comprises at least one of PVP and a PVP derivative.

71. A method of manufacturing a dosage form, which method comprises the steps of:
providing a liquid comprising a biologically compatible polymer;
supplying the liquid to a liquid supply tube having an outlet;
establishing an electric field between the outlet and a surface spaced from the outlet to cause liquid issuing from the outlet to form a jet which dries to form a polymer fibre which deposits onto the surface to form a fibre body; and
subjecting the fibre body to further processing to define a discrete dosage form shaped to be received within a nasal passage, the fibre being arranged to dissolve or disintegrate when the discrete dosage form is placed within a nasal passage.

72. A method according to claim 71, which comprises carrying out the further processing of the fibre body by cutting a cylindrical dosage form from the fibre body.

73. A method according to claim 72, which further comprises rounding an insertion end of the cylindrical dosage form.

74. A method according to any one of claims 71 to 73, which further comprises providing at least one through aperture through the dosage form to enable a user to breathe through the dosage form when the dosage form is first inserted into a nasal passage.

75. A method according to any one of claims 71 to 74, wherein the body carries at least one active ingredient that is at least one of: dispersed it the body as particles, dissolved in the material forming the body, provided on the outer surface of material forming the body.

76. A nasal dosage form manufactured by a method in accordance with any one of claims 71 to 75.
